(19) United States
(12) Patent Application Publication
(10) Pub. No.: US 2011/0251440 A1
(43) Pub. Date: Oct. 13, 2011

(54) METHOD AND APPARATUS FOR PRESSURIZING AND HEAT-TREATING A FLOWABLE SUSPENSION

(75) Inventors: Thomas Huegle, Worms (DE); Ralf Weigel, Bobenheim (DE)

(73) Assignee: DEMETRION DEUTSCHLAND AG, Kaiserslautern (DE)

(21) Appl. No.: 13/081,512

(22) Filed: Apr. 7, 2011

Related U.S. Application Data
(60) Provisional application No. 61/322,587, filed on Apr. 9, 2010.

(51) Int. Cl. C07C 31/08; B01J 3/00; C07C 4/00

(52) U.S. Cl. 568/840; 585/700; 422/242

(57) ABSTRACT

A method of continually heat-treating a biogenic material in the production of fuels such as ethanol or biogas includes pressurizing the biogenic material with a first pump so as to provide a pressurized suspension. The pressurized suspension is continuously heat-treated at a heat-treatment temperature of from 100° C. to 180° C. in a hydrolysis reactor so as to provide a heat-treated suspension. A temperature of the heat-treated suspension is lowered to below 100° C. The heat-treated suspension is depressurized with a second pump. The first and second pumps are configured to maintain at least one of the pressurized suspension and the heat-treated suspension at a pressure of above 5 bar.

Priority is claimed to U.S. patent application Ser. No. 61/322,587, filed Apr. 9, 2010. The entire disclosure of said application is incorporated by reference herein.

### FIELD

The present invention provides a method of heat-treating a flowable suspension, for example, a suspension comprising biogenic material or other organic material, the method comprising the steps of pressurizing the suspension, heat-treating the pressurized suspension, and depressurizing the heat-treated suspension by means of a fixed flow rate pump. The present invention also provides a device for subjecting a flowable suspension, for example, a suspension comprising biogenic material, to a temperature hydrolysis, the device comprising means for pressurizing the suspension, means for heat-treating the pressurized suspension and a fixed flow rate pump for depressurizing the suspension.

### BACKGROUND

There are many occasions in which a heat-treatment of a suspension is desirable. In particular, this concerns the processing of suspensions of biogenic or other organic material. These suspensions, can for example, be sterilized by heat-treatment. Heat treatment can also be an important step in processes that convert organic or biogenic material into other useful products such as ethanol or biogas. Exposing the organic or biogenic material to elevated temperatures can, for example, induce hydrolysis of complex macromolecules. Methane-containing biogas can, for example, be generated by anaerobic microbial digestion of biogenic material in a fermentation vessel. In multiple steps, the microorganisms break down the complex macromolecules of the biogenic material, thereby generating biogas comprising methane, carbon dioxide, water and other gaseous molecules. The biogas is subsequently cleaned and then converted into electrical and thermal energy in a combustion engine.

In general, of the many steps performed by the microorganisms in the fermentation vessel, the hydrolysis step is the slowest and therefore determines the overall speed of anaerobic microbial degradation. In order to provide for faster hydrolysis, it has been suggested to subject the biogenic material to a temperature hydrolysis step before introducing it into the fermentation vessel. It has in particular been found that the hydrolysis of cellulose and hemicellulose can be greatly expedited by the application of high temperatures.

The heat treatment can be performed by exposing the flowable suspension to an elevated temperature which, may be above 100° C. In order to prevent the liquid fraction of the suspension from vaporizing at temperatures at or above 100° C., the suspension must be pressurized. Once the heat treatment step is completed, the suspension can be cooled down again and it can be depressurized.

Use of a valve or a cascade of valves has been described for depressurizing the suspension. The use of spring-charging such valves has also be described so that the opening of the aperture is proportional to the pressure of the suspension in order to regulate the pressure. Alternatively, an electronically controlled valve has been proposed.

A disadvantage of the depressurizing valves is that they tend to clog. It is assumed that this is partly due to the very irregular distribution of particle sizes which is typical for a suspension of biogenic material and partly due to the fact that the aperture of the valve must be kept relatively small because the suspension's compressibility is very low so that even small amounts of suspension passing through the valve can lead to a disproportioned drop in pressure. In fact, a significant difficulty is to maintain the suspension within the desired pressure range using valves. Sudden pressure drops may thereby occur, entailing undesirable steam hammering. It has also been observed that the valves can quickly abrade, probably due to the solid fraction of the suspension and the high speed with which the suspension passes through the valve.

DE 26 45 150 describes a method of treating slurries in which the slurries are heated, pressurized and brought to a boil. The boiled slurry is then concentrated in a filter and fed into an incinerator. In order to limit the delivery rate into the incinerator, employing a dosing pump, such as an eccentric rotary pump or a peristaltic pump, is described.

### SUMMARY

An aspect of the present invention is to provide an improved method of heat-treating a flowable suspension, which overcomes drawbacks described in the prior art. A further, alternative aspect of the present invention is to provide an improved device for heat-treating a flowable suspension, which device overcomes drawbacks of known devices.

In an embodiment, the present invention provides a method of continually heat-treating a biogenic material in the production of fuels such as ethanol or biogas which includes pressurizing the biogenic material with a first pump so as to provide a pressurized suspension. The pressurized suspension is continuously heat-treated at a heat-treatment temperature of from 100° C. to 180° C. in a hydrolysis reactor so as to provide a heat-treated suspension. A temperature of the heat-treated suspension is lowered to below 100° C. The heat-treated suspension is depressurized with a second pump. The first and second pumps are configured to maintain at least one of the pressurized suspension and the heat-treated suspension at a pressure of above 5 bar.

### BRIEF DESCRIPTION OF THE DRAWINGS

The present invention is described in greater detail below on the basis of embodiments and of the drawings in which:

FIG. 1 shows a simplified process flow diagram of a method and device according to the present invention; and

FIG. 2 shows a simplified process flow diagram of the conduit system of FIG. 1.

### DETAILED DESCRIPTION

In an embodiment, the present invention provides a method of heat-treating a flowable suspension, the method comprising the steps of pressurizing the suspension, heat-treating the pressurized suspension, and depressurizing the heat-treated suspension by means of a fixed flow rate pump, wherein the fixed flow rate pump is controlled to keep the pressured suspension essentially within a pre-determined pressure range.

In an embodiment, the present invention provides a device for heat-treating a flowable suspension, the device comprising means for pressurizing the suspension, means for heat-treating the pressurized suspension and a fixed flow rate pump for depressurizing the suspension, wherein the device comprises control means for controlling the depressurizing pump such that the pressure of the pressurized suspension is kept within a pre-determined pressure range.

Advantageously, in the fixed flow rate pump according to the present invention, the volumetric flow rate is essentially independent of the pressure of the suspension before and/or after the pump. In other words, it essentially lacks the volume-pressure-elasticity observed in common kinetic pumps such as eductor-jet pumps. Examples for fixed flow rate pumps are progressive cavity pumps, gear pumps, including internal and external gear pumps and georotors, vane pumps, piston pumps, lobe pumps, and peristaltic pumps.

It is an achievable advantage of the present invention that the pressure of the suspension can be kept within a pre-determined range. For example, the pressure can be kept high enough to avoid vaporization of a liquid component of the suspension. The pressure can thereby moreover be kept low enough to ensure the safety of the device and avoid a waste of energy.

It is another achievable advantage of the present invention, that clogging can be avoided. Finally, abrasion as observed in the valves of the prior art devices can be avoided, thereby reducing maintenance costs.

The present invention can, for example, be used to sterilize biogenic material or to subject the flowable suspension to a temperature hydrolysis, for example, in order to pretreat biogenic or other organic material in the production of fuels such as ethanol or biogas.

Embodiments of the present invention, which may be applied alone or in combination, are disclosed in the dependent claims. Reference numerals provided herein are not meant to limit the scope of the invention to specific embodiments, but merely serve to facilitate reading of the claim by means of reference to such exemplary embodiments.

The flowable suspension can, for example, be an aqueous suspension. An example is a suspension of organic material, such as biogenic material. At least the solid component of the suspension can, for example, comprise or consists of biogenic or other organic material. The biogenic material can, for example, be reduced to small pieces, such as in a mill or a macerator, and mixed with water or, for example, with recyclate coming from a fermenter, or with a liquid component of the recyclate obtained by means of a separation device. The biogenic material may be renewable raw material, for example, renewable vegetable raw material such as corn, sugar-beet, sugar-cane, straw or wood. It may also be a biogenic residue such as organic industrial or agricultural waste, sewage sludge, slaughter waste, kitchen slops, food leftovers and adulterated food stuff. The present invention can be employed advantageously with biogenic material comprising cellulose and/or hemi-cellulose and/or lignin. A fraction of biogenic material in the suspension can, for example, be between 2 and 30%, for example, between 4 and 20%, for example, between 8 and 15%, or for example, between 10 and 13%, in terms of dry mass of biogenic material.

When the suspension is depressurized by means of the depressurizing pump, it sets the pump in motion, for example, in rotation. The pressure of the suspension can, for example, be controlled by means of controlling the rate of motion, such as the rate of rotation, of the depressurizing pump.

In an embodiment, the device according to the present invention can be provided with a pressure sensor to measure the pressure of the pressurized suspension. The rate of motion can, for example, be controlled in relation to the pressure of the suspension as measured by the pressure sensor. The skilled person will be aware of appropriate methods of controlling the pump's motion, for example, via the use of brakes. In an embodiment of the present invention, the rate of motion can, for example, be controlled by controlling an electrical generator, which generator is driven by the motion of the depressurizing pump. The generator may, for example, be controlled by adjusting a load applied to the generator. Thus, by means of the generator, the flow through the depressurizing pump can be controlled such that deviations from the desired pressure can be compensated for so that in particular undesired pressure drops can be avoided.

In an embodiment of the present invention, the lower limit of the pressure range can, for example be above 5 bar, for example, above 10 bar, for example, above 20 bar, or for example, above 25 bar. In an embodiment of the present invention, the lower limit of the pressure range may be above 30 bar. With such pressures, vaporization can be avoided even at relatively high temperatures. The upper limit of the pressure range can, for example, be below 100 bar, for example, below 70 bar, for example, below 50 bar, for example, below 40 bar, or for example, below 35 bar. The pressure range can, for example, have a width of 40 bar or less, for example, 30 bar or less, for example, 20 bar or less, or for example, 15 bar or less. The pressure range can, for example, be between 10 to 25 bar, between 12 to 20 bar, or between 15 and 17 bar. In an embodiment of the present invention, the suspension enters the pressurizing pump at approximately atmospheric pressure. The suspension can, for example, leave the depressurizing pump at a pressure still above atmospheric pressure so that the remaining overpressure can be used to further transport the suspension, such as to a fermentation vessel. In an embodiment of the present invention, the suspension can leave the depressurizing pump at a pressure of approximately 2 bar.

The depressurizing pump can, for example, provide for a steady delivery flow. In other words, the pump provides for a delivery flow that is essentially constant over the course of a full pump cycle. Pumps that can achieve a steady delivery flow include, for example, progressive cavity pumps and vane pumps. According to an embodiment of the present invention, oscillations in the pressure can be avoided, which oscillations may otherwise lead to undesirable pressure drops and steam hammering.

In an embodiment of the present invention, the depressurizing pump can, for example, be a progressive cavity pump, which is also sometimes referred to as an eccentric screw pump. An advantage of this embodiment of the present invention that a very steady delivery flow can be achieved. A low friction between the suspension and the pump can also be achieved, which reduces wear of the pump. The progressive cavity pump can also be employed at relatively high pressures. In the progressive cavity pump, the suspension can be transferred by means of a progress, through the pump, of a sequence of fixed shape, discrete cavities as a rotor of the pump is turned. The volumetric flow rate can, for example, be proportional to the rate of rotation of the rotor. The rotor can, for example, be of stainless steel, while the stator can, for example, comprise a moulded elastomer.

In an embodiment of the present invention, pressurizing of the suspension can, for example, be performed by a pressurizing pump. The pressurizing pump can, for example, be a fixed flow rate pump, such as a fixed flow rate pump that provides for a steady delivery flow. An example of a pressurizing pump is a progressive cavity pump, also referred to as an eccentric screw pump. As in the ease of the depressurizing pump, with this embodiment of the present invention, oscillations can be avoided which may otherwise lead to undesirable pressure drops and/or steam hammering. The wear of the pump is relatively low due to the low friction between the suspension and the pump. The progressive cavity pump can also be employed at relatively high pressures.

When set into motion by the suspension, the depressurizing pump generates mechanical energy. This energy can be harvested, for example, by means of an electric generator. In an embodiment of the present invention, the energy generated by the depressurizing pump can contribute to drive the pressurizing pump. In other words, at least part of the energy consumed by the pressurizing pump can be recovered by means of the depressurizing pump. This may be achieved, for example, by supplying at least part of the energy generated by an electrical generator driven by the depressurizing pump to an electrical motor driving the pressurizing pump. Energy may also be transferred mechanically from the depressurizing pump to the pressurizing pump, for example, by means of a belt and/or a rotary shaft and/or a gearing. In general, the energy generated by the depressurizing pump will not be sufficient to drive the pressurizing pump. The pressurizing pump is, at least in addition to the energy from the depressurizing pump, supplied with energy from another source.

If an electric generator is used to convert the mechanical energy generated by the depressurizing pump into electricity, this electricity may also be at least partly be fed into an external electricity network, such as a local or regional electricity grid, which would then in turn indirectly supply energy to the pressurizing pump.

The method according to the present invention can, for example, be carried out as a continuous process. In an embodiment of the device according to the present invention, the pressurizing pump and the depressurizing pump can be connected by a conduit system through which the pressurized suspension is passed to be heat-treated. The conduit system can, for example, comprise a reactor to perform the heat treatment, such as a hydrolysis reactor, to subject the suspension to a temperature hydrolysis. The conduit system can also comprise a heater and/or heating means for heating up the suspension after it has been pressurized and a cooler and/or cooling means for cooling the suspension down again before it is depressurized. In an embodiment of the present invention, the heating and/or cooling devices and/or means are coupled for heat recovery, so that at least part of the heat absorbed in the cooling means can be used in the heating means for heating up the suspension.

The suspension can, for example, be continuously passed through the conduit system. The process can, for example, be performed in a mode in which essentially the same amount of suspension enters the conduit system through the pressurizing pump as suspension leaves the conduit system through the depressurizing pump.

In the method according to the present invention, the step of heat-treating the suspension comprises subjecting the suspension to a heat-treatment temperature of above 100° C., for example, above 140° C., for example, above 180° C., for example, above 220° C., or for example, above 240° C. The temperature can, for example, be from between 140° C. to 170° C., or from 150° C. to 160° C. The suspension can, for example, be maintained at this temperature for a predetermined time, for example, in the hydrolysis reactor. The predetermined time can, for example, be at least 5 minutes, at least 10 minutes, at least 15 minutes, at least 20 minutes, at least 25 minutes, at least 30 minutes, or at least 35 minutes. The predetermined time can, for example, be from 15 to 25 minutes, or from 18 to 22 minutes, or for about 20 minutes. The suspension can, for example, be maintained at the treatment temperature long enough to provide the statutory hydraulic retention time for the sterilization of biogenic material, such as according to the pertinent European Community hygiene directive.

After subjecting the present invention to the hydrolysis temperature, the temperature can, for example, be lowered again to a temperature below 100° C., for example, below 80° C., below 70° C., or below 60° C. The step of lowering the temperature can, for example, take place before the step of depressurizing the heat-treated suspension by means of the fixed flow rate pump.

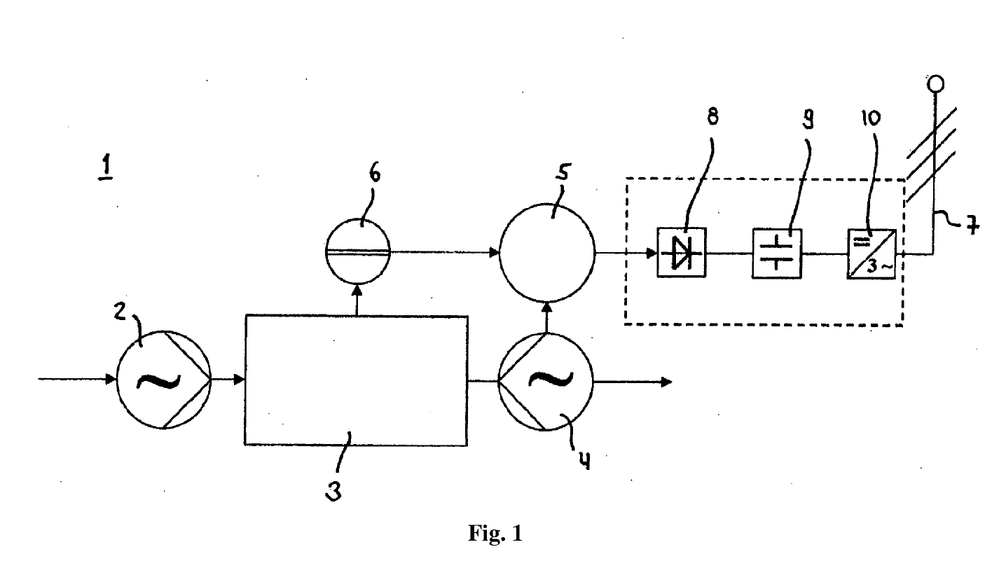

An embodiment of the device 1 according to the present invention is illustrated in FIG. 1 by means of a simplified process flow diagram. Biogenic material has been reduced to small pieces in a mill or in a macerator (not shown) and has then been suspended in water or the liquid component of an aqueous recirculate coming from a fermenter (not shown) to form a suspension comprising about 10 to 15% biogenic material (with respect to the dry mass of the biogenic material). The liquid component has been obtained by separating it from the remainder by means of a separation device (not shown).

The suspension, which originally is at atmospheric pressure, is pressurized by a progressive cavity pump 2 to a pressure within the interval from 25 to 40 bar. The compressed suspension is then introduced into the conduit system where it is subjected to a heat treatment. The treated, still pressurized suspension is then depressurized in another progressive cavity pump 4 to a pressure of 2 bar. This residual overpressure is used to further transport the suspension to the fermenter.

The progressive cavity pumps each comprise a helical rotor with a circular cross-section, which performs an eccentric rotary motion in a stator with an oval cross-section. The rotor is of stainless steel while the stator is of a moulded elastomer. As the rotor turns, suspension is transferred by means of the progress, through the pump, of a sequence of fixed shape, discrete cavities. The volumetric flow rate is directly proportional to the rate of rotation.

The flow rate of the depressurizing progressive cavity pump 4 is controlled to maintain the pressurized suspension within the narrow interval from 25 to 40 bar. The pressure is thereby kept high enough to avoid vaporization and at the same time low enough so as not to exceed the maximum pressure at which the device can operate safely according to its construction. For this purpose, the depressurizing pump 4 drives an electrical generator 5. By means of the generator 5, the flow through the depressurizing pump 4 can be controlled to compensate for deviations from the desired pressure. To achieve this, the load of the generator 5 is adjusted as a function of the pressure of the suspension as measured by a pressure sensor 6 which measures the pressure of the pressurized suspension in the hydrolysis reactor 3. When the load of the generator 5 is increased, the generator 5, and thus the depressurizing pump 4, are slowed down, thereby reducing the pump's 4 flow rate and thus increasing the pressure of the pressurized suspension in the conduit system 3. Undesired pressure drops can thereby be avoided.

The electricity generated by the electrical generator 5 in an embodiment of the present invention is introduced into an external electricity network 7 after being rectified in a rectifier 8, filtered in a filter 9, and converted by a converter 10 into the required direct or alternating, e.g. three-phase, voltage.

In an embodiment of the present invention, all or at least part of the electricity generated by the generator 5 is, for example, also after rectification, filtering and conversion, used to drive a motor (not shown), which motor then drives the pressurizing pump 2.

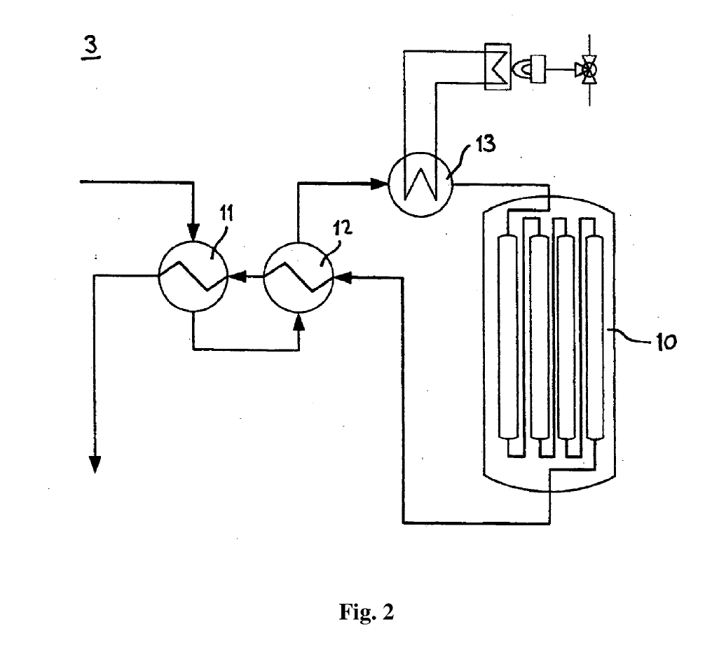

FIG. 2 illustrates by means of a simplified process flow diagram in greater detail the conduit system 3 through which the pressurized suspension is passed. The pressurized suspension coming from pressurizing pump 2 has approximately room temperature. Before being introduced into reactor section 10, it passes through two heat exchangers 11, 12 in which it takes up heat from suspension leaving the reactor section 10. The suspension is moreover led through another heat exchanger 13, which uses thermal oil heated by a heat source 14 or waste heat of a combustion engine (not shown) run on the biogas coming from the fermenter. As a result, the suspension has a temperature of approximately 200° C. when it enters the reactor section 10.

The volume of the reactor section is chosen to provide a statutory hydraulic retention time of, for example, at least 20 minutes. Leaving the reactor section 10, the suspension is cooled down again as it passes through the heat exchangers 12, 11 giving off much of its heat to the incoming suspension. The suspension leaves the hydrolysis reactor at a temperature of approximately 70° C.

The present invention is not limited to embodiments described herein; reference should be had to the appended claims.

What is claimed is:

1. A method of continually heat-treating a biogenic material in the production of fuels such as ethanol or biogas, the method comprising the steps of:
pressurizing the biogenic material with a first pump so as to provide a pressurized suspension;
continuously heat-treating the pressurized suspension at a heat-treatment temperature of from 100° C. to 180° C. in a hydrolysis reactor so as to provide a heat-treated suspension;
lowering a temperature of the heat-treated suspension to below 100° C.; and
depressurizing the heat-treated suspension with a second pump, wherein the first and second pumps are configured to maintain at least one of the pressurized suspension and the heat-treated suspension at a pressure of above 5 bar.

2. The method as recited in claim 1, wherein a pressure of the pressurized suspension is controlled by the second pump.

3. The method as recited in claim 1, wherein the pressure is from 10 to 25 bar.

4. The method as recited in claim 1, wherein the pressure is from 15 to 17 bar.

5. The method as recited in claim 1, wherein the heat-treatment temperature is from 150° C. to 160° C.

6. The method as recited in claim 1, wherein the temperature is lowered to below 80° C.

7. The method as recited in claim 1, wherein the second pump is configured to provide a continuous delivery flow.

8. The method as recited in claim 1, wherein at least one of the first pump and the second pump is a progressive cavity pump.

9. The method as recited in claim 1, wherein the second pump is configured to generate an energy which is used to drive the first pump at least one of directly or indirectly.

10. A device for heat-treating a biogenic material according to the method recited in claim 1, the device comprising:
a first pump configured to pressurize the biogenic material;
a hydrolysis reactor configured to heat-treat a pressurized suspension;
a second pump configured to depressurize the pressurized suspension; and
control devices configured to control the second pump so as to maintain a pressure of the pressurized suspension of above 5 bar.

11. The device as recited in claim 10, wherein the first pump is a pressurizing pump and the first pump and the second pump are connected by a conduit system through which the pressurized suspension to be heat-treated is passed.

12. The device as recited in claim 10, wherein the pressure is from 10 to 25 bar.

13. The device as recited in claim 10, wherein the pressure is from 15 to 17 bar.
